(19) United States
(12) Patent Application Publication
(10) Pub. No.: US 2008/0015578 A1
(43) Pub. Date: Jan. 17, 2008

(54) Orthopedic implants comprising bioabsorbable metal

(76) Inventors: Dave Erickson, Memphis, TN (US); Naim Istephanous, Roseville, MN (US); Paul J. Wisnewski, Maple Grove, MN (US); Nelson Oi, Memphis, TN (US); Heather Savage, Memphis, TN (US)

Correspondence Address: KRIEG DEVAULT LLP, ONE INDIANA SQUARE, SUITE 2800, INDIANAPOLIS, IN, 46204-2709, US

(21) Appl. No.: 11/485,559

(22) Filed: Jul. 12, 2006

(51) Int. Cl. A61F 2/30

(52) U.S. Cl. 606/61

(57) ABSTRACT

An implantable device comprises at least one component composed of a bioabsorbable metal. The component has desirable structural/mechanical properties upon implant, and then begins to degrade at a time after implant, and is absorbed partially or completely over time.

### BACKGROUND

The present invention relates to the field of biomedical implants and, in particular, implantable devices comprising a bioabsorbable metal. The devices have certain desired structural/mechanical properties upon implant, and then all or portions thereof begin to degrade at a time after implant through controlled kinetics, and are absorbed partially or completely without requiring further surgery for removal.

The use of orthopedic implants, bone grafts and bone substitute materials in orthopedic medicine is well known. While bone wounds can regenerate, fractures and other orthopedic injuries take a substantial time to heal, during which the bone is unable to support physiologic loads. It is well understood that stabilization of adjacent bony portions can be completed with an implant positioned between the bony portions and/or an implant positioned along the bony portions. The implants can be rigid to prevent motion between the bony portions, or can be flexible to allow at least limited motion between the bony portions while providing a stabilizing effect. As used herein, bony portions can be portions of bone that are separated by one or more joints, fractures, breaks, or other space.

Metallic materials have played an essential role as biomaterials to assist with the repair or replacement of bone tissue that has become diseased or damaged. For example, metal pins, screws, plates, rods, and meshes are frequently required to replace the mechanical functions of injured bone during the time of bone healing and regeneration. Metals are more suitable for load-bearing applications compared with ceramics or polymeric materials due to their combination of high mechanical strength and fracture toughness. Currently approved and commonly used metallic biomaterials include stainless steels, titanium and cobalt-chromium-based alloys. One limitation of these current metallic biomaterials is that their elastic moduli are not well matched with that of natural bone tissue. These metals are significantly stiffer than bone, which results in stress shielding effects that can lead to reduced stimulation of new bone growth and remodeling and decreased bone density around the implant site, both of which decrease implant stability.

The structural requirements placed upon orthopedic devices are even more pronounced when considering implants that are required to provide structural support to a human spine. For example, spinal fusions require interbody fusion devices that will maintain significant structural rigidity for at least 6-12 months, and strength requirements depend on the location of the disc to be replaced. When a person is standing, the forces to which a disc is subjected are much greater than the weight of the portion of the body above it. It has been reported that the force on a lumbar disc in a sitting position is more than three times the weight of the trunk. Products designed for chronic fixation and support of the spine segments and fusion of the spine are most commonly fabricated from metallic alloys such as Ti-6AI-4V or 316LVM SS, which are known for their biocompatibility and mechanical strength. These materials are desirable for such use because of their proven performance in medical implant applications, mechanical properties, good biocompatibility, availability in a range of forms, and good corrosion resistance.

While current metallic biomaterials are essentially neutral in vivo, and often remain as permanent fixtures, such mechanical constructs are often only needed for a relatively short period of time such as, for example, during a period that is sufficient to provide support during the progress of natural healing. In the case of plates, screws and pins used to secure serious fractures, these devices often must be removed by a second surgical procedure after the tissue has healed sufficiently. Repeat surgery increases costs to the health care system and further morbidity to the patient. Other metallic implants are allowed to simply remain at the healing site after healing has occurred and the need for the metal implant has passed, which is also less than ideal. Leaving a metallic implant in place after graft fusion has been reported to increase the risk of adjacent disc disease. To address this issue, alternative materials such as bioabsorbable polymers have been investigated. These materials are attractive in that they are resorbed over time, i.e., are eventually degraded and at least partially removed from the body by natural processes. However, these materials have been found to lack sufficient strength and creep resistance for applications involving significant tensile, torsional, or bending loads, and their use has been limited to compressive load and non-load-bearing applications.

It is apparent from the above that there is a continuing need for advancements in the relevant field, including new implant and device designs and new material compositions and configurations for use in medical devices. The present invention is such an advancement and provides a variety of additional benefits and advantages.

### BRIEF DESCRIPTION OF THE DRAWINGS

FIG. 1 is a sectional view of an implant assembly according to one embodiment.

FIG. 2 is an elevation view of a spinal column segment with a pair of implant devices secured thereto.

FIG. 3 is an elevation view of another embodiment implant component.

FIG. 4 is a sectional view of a portion of the implant component of FIG. 3 with an anchor for securing the component to a bony portion.

FIG. 5 is a longitudinal cross-sectional view of a spinal rod embodiment.

FIG. 6 is an elevation view of an end of another spinal rod embodiment.

FIG. 7 is an elevation view of an end of another spinal rod embodiment.

FIG. 8 is a partial longitudinal cross sectional view of another spinal rod embodiment.

FIG. 9 is an elevation view of an end of another spinal rod embodiment.

FIG. 10 is an elevation view of an end of another spinal rod embodiment.

FIG. 11 is a partial longitudinal cross sectional view of another spinal rod embodiment.

FIG. 12 is a partial longitudinal cross sectional view of another spinal rod embodiment.

FIG. 13 is a perspective view of one embodiment of a bone fixation device comprising an elongate member in the form of a bone plate in accordance with the invention.

### SUMMARY

The present invention involves orthopedic implant devices made using one or more bioabsorbable metals. Inventive devices provide a desirable alternative material for orthopedic implants, combining sufficient strength and stiffness to provide the necessary support for spinal fixation and stabilization, with the capability to resorb in a controlled manner over time.

The invention provides in one aspect an orthopedic implant device that comprises a bioabsorbable metal composition, and comprises at least two different components that exhibit different degradation profiles after implant. In one embodiment, the device comprises at least two structural components that are composed of different bioabsorbable metal compositions that exhibit different absorption profiles after implant. In another embodiment, the device comprises at least two structural components that are composed of a bioabsorbable metal composition, at least one of which has a coating thereon composed of a bioabsorbable composition; wherein the structural components exhibit different absorption profiles after implant due to the presence of the coating. The at least two structural components can be composed of the same bioabsorbable metal composition or different bioabsorbable metal compositions. In one embodiment, at least two of the structural components have different coatings thereon, each coating composed of one or more bioabsorbable composition; and the components exhibit different absorption profiles under a given set of conditions due to the presence of the coatings.

In another embodiment of the invention, the device comprises at least one bone-engaging element and at least one non-bone-engaging element, and the absorption profiles of the bone-engaging element and the non-bone-engaging element are selected to ensure that absorption of the non-bone-engaging element is completed before the occurrence of significant degradation of the bone-engaging element. The bone-engaging element can be, for example, a bone screw or an anchor. The non-bone-engaging element can be, for example, a rod, a bracket or a plate. In another embodiment, all components of the device are absorbed within a five-year period.

In yet another embodiment, at least one component of the device comprises a bioactive material such as, for example, an osteoconductive or osteoinductive bioactive material, impregnated in the component or applied to the component as a surface treatment. In one embodiment, at least one component comprises a bioabsorbable metal compounded with the bioactive material. Alternatively, a component can be manufactured to be porous, and a bioactive material can optionally be impregnated therein or coated thereon after the porous component is formed. In another embodiment, at least one component comprises a bioabsorbable metal compounded with a bioabsorbable polymer.

In another aspect of the invention, there is provided an orthopedic implant device that comprises at least two different metals, and wherein at least one of the metals is a bioabsorbable metal composition. In one embodiment, the device includes a spinal rod that includes a main spinal rod body, the main spinal rod body composed of a first metal and defining at least one internal chamber; and a core positioned within the internal chamber, the core composed of a second metal that is a bioabsorbable metal. In one embodiment, the device also includes a source of electrical potential operably connected to the core. The source can be a battery, for example. In another preferred embodiment, the core is coupled to the main spinal rod body. In yet another embodiment, a channel is formed between a surface of the main spinal rod body and the core to provide a conduit for infiltration of body fluid after implantation. Alternatively, the main spinal rod body can define a plurality of internal chambers, and the device can include a plurality of core members positioned within some or all of the internal chambers. The core members can be composed of different bioabsorbable metal compositions or can be composed of the same bioabsorbable metal compositions. The device can also include a plurality of caps, plugs or seals operable to shield at least one of said core members from contact with body fluid after implant. In still another embodiment, the main spinal rod body defines an internal chamber, and a plurality of core members are positioned within the internal chamber.

In another aspect of the invention, there is provided an orthopedic implant device that comprises: (1) an elongate member including a receptacle therein configured to be fixedly secured to two or more bone portions allowing translational or rotational, or both translational and rotational movement of a first one of the bone portions relative to a second one of the bone portions; and (2) a restricting component composed of a bioabsorbable metal composition and disposed in the receptacle to inhibit the translational, the rotational, or both the translational and rotational movement of the first of the bone portions relative to the second of the bone portions. The device can be configured such that the elongate member allows limited translational, or rotational, or translational and rotational movement of the first of one of said two or more bone portions relative to the second of said two or more bone portions after the restricting component biodegrades.

In another aspect, the invention provides an orthopedic implant device that comprises a bioabsorbable metal composition, and comprises at least two different components that exhibit different degradation profiles after implant, wherein degradation of the bioabsorbable metal composition is initiated or enhanced by an electrical potential across the bioabsorbable metal composition. The device can include an electrical potential source operably connected to the bioabsorbable metal composition.

The invention provides in another aspect an orthopedic implant device comprising at least one structural component constructed from a bioabsorbable metal composition, the structural component having physical properties effective to withstand tensile loads, torsional loads and bending loads encountered during spinal implant procedures and during a first period of time of at least 6 months post-implant, and the structural component being absorbed within a second period of time. In one embodiment, the first and second periods of time begin when the device is surgically implanted; the first period of time ends after bone repair or fusion has proceeded to a degree where the physical properties of the component are no longer required; and the second period of time is greater than the first period of time. The second period of time is preferably less than three years. The bioabsorbable metal composition can comprise a member selected from the group consisting of magnesium, iron, a magnesium-based alloy and an iron-based alloy. In one embodiment, the bioabsorbable metal composition is a magnesium-based alloy comprising at least about 85% magnesium by weight and an alloying element portion comprising an element selected from the group consisting of aluminum, zinc, a rare earth element, manganese, lithium, zirconium and yttrium. In another embodiment, the bioabsorbable metal composition is an iron-based alloy comprising at least about 85% iron by weight and an alloying element portion comprising an element selected from the group consisting of aluminum and magnesium.

In one embodiment, the device also includes a coating component effective to prevent, or control the kinetics of, degradation of the bioabsorbable metal composition during some or all of the first period of time. Breach of the coating initiates degradation of the structural component. The coating can comprise a bioabsorbable composition, such as, for example, a bioabsorbable composition selected from the group consisting of a bioabsorbable metal composition and a bioabsorbable polymeric composition. The bioabsorbable composition can also have a bioactive agent impregnated therein. In one embodiment, the bioabsorbable composition is selectively degradable, thereby providing for controlled removal of the coating and initiation of degradation of the component. In one preferred embodiment, degradation of the coating is initiated by application of an electrical current to the coating.

In another aspect of the invention, an orthopedic implant product includes at least two different components that exhibit different degradation profiles under a given set of conditions, one of said components composed of a bioabsorbable metal composition, wherein degradation of the bioabsorbable metal composition is initiated or enhanced by an electrical potential applied to the bioabsorbable metal composition; and wherein the product also includes instructions, recorded in a tangible medium, regarding applying an electrical potential across the bioabsorbable metal composition. The instructions can include instructions for synchronizing the application of electrical potential, and thus the initiation of degradation, to a desired stage of healing or fusion.

The invention provides in another aspect a method for treating a bone defect comprising fixedly attaching the device of claim 1 to two or more bone portions. In yet another aspect, the invention provides a method for treating a bone defect that includes: (1) providing an orthopedic implant device comprising a bioabsorbable metal composition, and comprising at least two different components that exhibit different degradation profiles under a given set of conditions; (2) securing the device to first and second bone portions; and (3) allowing the biodegradable metal composition to degrade in vivo.

An object of the present application is to provide a unique orthopedic implant device.

Further embodiments, forms, features, aspects, benefits, objects, and advantages of the present application shall become apparent from the detailed description and figures provided herewith.

### DETAILED DESCRIPTION OF SELECTED EMBODIMENTS

For the purpose of promoting an understanding of the principles of the invention, reference will now be made to the embodiments set forth herein and specific language will be used to describe the same. It will nevertheless be understood that no limitation of the scope of the invention is thereby intended. Any alterations or further modifications of the described embodiments and any further applications of the principles of the invention as described herein are contemplated as would normally occur to one skilled in the art to which the invention relates.

The present invention provides implantable medical devices that include at least one bioabsorbable metal composition. As used herein, the term “bioabsorbable” is intended to be interchangeable with the terms “biodegradable,” “bioerodable” and “resorbable” and to refer to materials that degrade under physiological conditions, with or without application of external forces, to form a product that can be metabolized or excreted (i.e., absorbed) without damage to organs. Biodegradable materials may be degradable, for example, by hydrolysis or oxidation, and may require cellular and/or enzymatic action to fully degrade. Biodegradable materials also include materials that are broken down within cells.

Inventive devices that include at least one bioabsorbable metal compositions find advantageous use in a variety of different circumstances in which it is desirable for some or all of a medical implant to be degraded and absorbed after a time period has passed during which its presence is required or desired. Degradation and absorption obviate the need for surgical removal. Specific examples of medical devices that are included within the scope of the present invention include, without limitation, orthopedic implants such as spinal implants that are employed alone or with other components to stabilize one or more vertebral levels. The medical device may be, for example, an intervertebral prosthesis, intravertebral prosthesis, or extravertebral prosthesis such as a bone plate, spinal rod, rod connector, or bone anchor. The invention is particularly advantageous for use in connection with fixation implants, such as, for example, anterior plates and screws, interbody fusion implants, such as cages, and components used in connection therewith, such as, for example, screws and anchors. As will be appreciated, a component for use in the spine is fabricated to exhibit suitable strength to withstand the biomechanical stresses and clinically relevant forces without permanent deformation. Other orthopedic implants, and other non-orthopedic medical devices are also contemplated by the invention. For orthopedic devices that are not implanted in or around the spine, the component can be fabricated to withstand the biomechanical forces exerted by the associated musculoskeletal structures. The medical devices can be used to treat a wide variety of animals, particularly vertebrate animals and including humans.

In one aspect of the invention, there is provided an orthopedic implant device comprising a bioabsorbable metal composition that includes at least two different components that exhibit different degradation profiles under a given set of conditions. The term “component” is used herein to refer a part of a device that is distinct from other parts by virtue of a different form and/or a different function and/or a different composition. It is to be understood that two different areas of a single unitary element or structure can be considered different components where, for example, the different areas have different properties or are composed of different compositions. For example, where a given structural component, such as, for example, a spinal rod, includes a main spinal rod body with a coating thereon that has different properties, the coating can be considered a different component than the main spinal rod body. In certain embodiments of the invention, therefore, implantable medical devices are provided that comprise a single element or structure that includes multiple components in an integral, unitary structure. The structure can be formed of metal and metal alloys that have been metallurgically joined at an atomic level by, for example, fusing or bonding, to provide an integral, unitary structure of at least two materials having differing performance characteristics along, about or within the structure.

When it is desired for a single element of a medical device to include more than one different metallic composition as different portions of the element, the element can be prepared using a variety of different processes. The medical devices can be formed to include one or more components having a material profile that includes, for example, a first metal or metal alloy that is fused, diffused, or bonded for joining at an atomic level with a second metal or metal alloy. In preferred embodiments, there is no need or requirement for a bonding layer between the first and second metals or metal alloys, although the use of a bonding layer is not precluded. It will be understood by those skilled in the art, however, that depending upon the method of fabrication, various zones, regions or diffusion layers may exist between the various materials comprising the component that could be considered to be a bonding layer. For the present invention, the term “bonding layer” is intended to mean that an intermediate layer, region or zone, that has materials that include at least in part both of the first and second materials comprising the component of the medical device and/or a layer of third material between the first and second materials. Preferred processes for forming a unitary component from multiple diverse metallic compositions include: conventional melting technology, such as, casting directional solidification, liquid injection molding, laser sintering, laser-engineered net shaping, powder metallurgy, metal injection molding (MIM) techniques; and mechanical processes such as rolling, forging, stamping, drawing, and extrusion. Also contemplated are cladding processes that can include cladding techniques; thermal spray processes that include: wire combustion, powder combustion, plasma flame and high velocity Ox/fuel (HVOF) techniques; pressured and sintered physical vapor deposition (PVD); chemical vapor deposition (CVD); or atomic layer deposition (ALD), ion plating and chemical plating techniques.

Metallic orthopedic implant devices in accordance with the invention can be fabricated to include at least two different metal compositions, at least one of which is a bioabsorbable metal composition. The term “metal composition” is used herein to refer to a composition composed entirely of an elemental metal or a combination of metal elements, as in the case of metal alloys. Inclusion of multiple metal compositions, including at least one bioabsorbable metal composition, in an implant device can provide a variety of advantageous features to a medical implant. For example, in circumstances where it would be desirable for an implant device to ultimately be absorbed in its entirety after its useful life has passed, uncontrolled simultaneous degradation of device components could lead to undesirable results such as, for example, premature separation of implant components from a patient's bone, as would occur if bone-engaging components were to degrade to a point of structural failure before substantial absorption of other components attached thereto is complete. Control of degradation of various parts of a device therefore can ensure that certain components remain structurally sound until after other components have been resorbed. This control can be exerted in one aspect of the invention by using different metal compositions to fabricate various device components, subcomponents or portions.

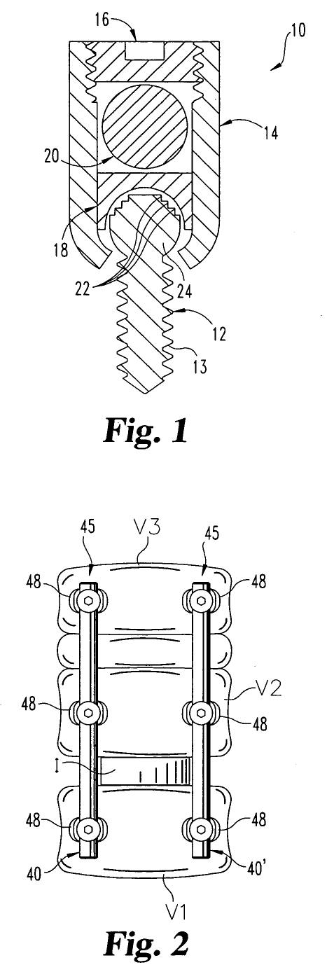

One exemplary application of inventive principles involves fabrication of a multi-axial spinal anchor, as shown in FIG. 1. Anchor 10 includes a bone engaging member 12, a receiver 14, an engaging member 16, and a load transfer member 18. Bone engaging member 12 can be pivotally mounted, engaged, or captured in receiver 14 so that a first bone engaging portion 13 thereof can assume any one of a number of angular orientations relative to receiver 14 and/or connecting member 20. Other embodiments contemplate a uni-axial arrangement between receiver 14 and bone engaging member 12.

An elongate connecting member 20, such as a spinal rod, can be positioned in receiver 14 between load transfer member 18 and engaging member 16. Engaging member 16 can be threadingly advanced along receiver 14 to secure connecting member 20 against load transfer member 18. Other embodiments contemplate that connecting member 20 can be positioned about or around receiver 14. It is also contemplated that engaging member 16 can be secured about or around receiver 14.

In the illustrated embodiment, load transfer member 18 is secured against bone engaging member 12 to secure bone engaging member 12 and connecting member 20 in position relative to one another. Bone engaging member 12 can include a head 24 with a number of ridges 22 extending thereabout. Load transfer member 18 engages the ridges 22 about head 24 or other suitable structure of bone engaging member 12 to lock bone engaging member 12 in position in receiver 14.

As will be appreciated by a person of ordinary skill in the art, full degradation and bioabsorption of bone engaging member 12 prior to degradation and bioabsorption of other components of multi-axial spinal anchor 10 or other implant components to which it is attached would result in such other components becoming detached from the bone. This could result in the other components separating from the bone, putting added stress on other points of fixation at the very least, which could cause pain and injury, and possibly allowing implant components to totally break away from the bone. As such, components of anchor 10 and associated implant components to which it is attached, are preferably fabricated in accordance with the invention to ensure that non-bone-engaging components, such as receiver 14, engaging member 16, load transfer member 18 and elongate connecting member 20 are able to be degraded and absorbed before the degradation of bone engaging member 12 proceeds to a point that it breaks free from the bone. Similarly, other elements can advantageously be composed of diverse bioabsorbable metal compositions to achieve chronological degradation in a desired manner.

Another exemplary implant device is bi-lateral spinal stabilization device 45 represented in FIG. 2 in which elongate stabilization elements 40, 40′ are secured along the spinal column with anchors 48 to first, second and third vertebrae V1, V2, V3 where no or very little motion between the vertebrae is desired, at least initially. One or more interbody implants I can be positioned in the disc space between vertebrae V1 and V2 for fusion of the vertebrae. Anchors 48 can be secured to respective ones of the vertebrae V1, V2, V3 to engage stabilization element 40 along the vertebrae. Anchors 48 can be multi-axial, uni-axial, or uni-planar screws; fixed angle bone screws; variable angle bone screws; staples; wires or cables; suture anchor and sutures; interbody devices; intrabody devices; and combinations thereof, for example, that are suitable to secure stabilization element 40, 40′ to the respective vertebrae. In addition, stabilization along three or more levels or stabilization of a single vertebral level is contemplated. In another embodiment, the stabilization element 40 can be secured along the spinal column with one or more of the anchors 10 discussed above.

As discussed above in connection with the multi-axial spinal anchor depicted in FIG. 1, full degradation of one or more of anchors 48 prior to degradation and bioabsorption of stabilization elements 40, 40′ could result in stabilization elements 40, 40′ becoming detached from the vertebrae V1, V2, V3. This would result in stabilization elements 40, 40′ becoming separated from the vertebrae, putting added stress on other anchors at the very least, which could result in pain and injury, and possibly allowing stabilization elements 40, 40′ to totally break away from the vertebrae. As such, components of bi-lateral spinal stabilization device 45 are preferably fabricated in accordance with the invention to ensure that non-bone-engaging components, such as stabilization elements 40, 40′ are degraded and absorbed before the degradation and absorption of anchors 48 proceeds to a point that stabilization elements 40, 40′ break free from the bone.

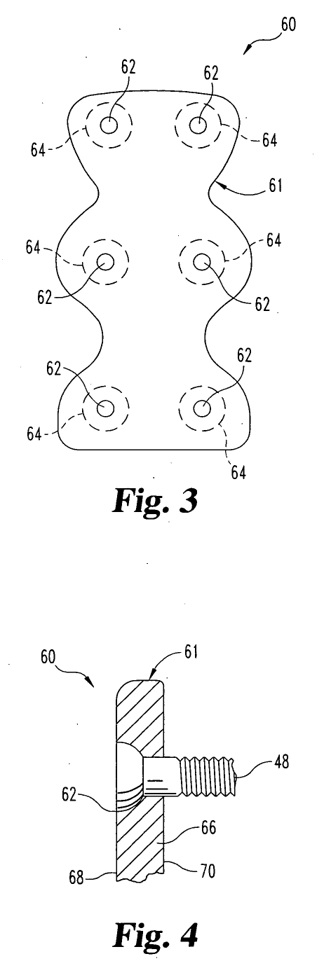

FIGS. 3 and 4 show another specific application for a medical device component including elongated stabilization element 60 in the form of a plate 61 that is attachable to at least two vertebrae of a spinal column. Plate 61 includes an elongated body having a number of holes 62 extending between upper and lower surfaces 68, 70 thereof to receive bone anchors 48 to secure plate 61 to the spinal column. In accordance with the invention, the plate and the anchors are preferably fabricated to ensure that degradation and absorption of the plate proceeds to completion before the degradation and absorption of anchors 48 proceeds to a point that plate 61 breaks free from the bone.

A variety of bioabsorbable metal compositions can be used in accordance with the present invention, provided that the selected bioabsorbable metal composition meets the functional requirements discussed herein. Selection of a particular bioabsorbable metal is based primarily on the known properties of the metal, such as, for example, its physical properties, its degradation profile, its biocompatibility and the like. The words “degradation profile” refer to the timing of and rate at which a component degrades and is absorbed by the body. For example, even two components composed of the same bioabsorbable metal composition can have different degradation profiles if degradation of one is caused to begin at a different time or to proceed at a different rate than the other. As used herein, the term “biocompatibility” refers to materials that, when implanted in a patient, do not induce undesirable long term effects. A preferred biocompatible material when introduced into a patient is not toxic or injurious to the patient, either as part of the bulk device or in particulate form, and does not cause immunological rejection. In particularly preferred embodiments, the metal materials include at least one material that has been accepted for use by the medical community, particularly the FDA and surgeons.

Multiple mechanisms exist by which a metallic component can be degraded into an absorbable form in accordance with the invention. In one manner of practicing the invention, the bioabsorbable metal selected for use in accordance with the invention is one that is degraded by natural corrosion in vivo that results from contact with body fluids. In another manner of practicing the invention, the bioabsorbable metal is one whose degradation is influenced by electrochemical means, such as, for example, via an external source of electric potential or via galvanic coupling.

One exemplary class of metals of the type that are degraded by natural corrosion in vivo that results from contact with body fluids is magnesium and its alloys. Magnesium is an exceptionally lightweight, and highly reactive, metal that is widely used in consumer product applications due to is combination of lightweight and strength characteristics. With a density of 1.74 g/cm3, magnesium is 1.6 and 4.5 less dense than aluminum and steel, respectively. The fracture toughness of magnesium is greater than ceramic biomaterials such as hydroxyapatite, while the elastic modulus and compressive yield strength of magnesium are closer to those of natural bone than is the case for other commonly used metallic implants.

Moreover, because magnesium is an essential element, implants composed of magnesium that are slowly degraded over time should not harm tissue, particularly since magnesium solutions up to 0.5 mol/l are well tolerated if given parenterally. Magnesium is essential to human metabolism and is naturally found in bone tissue. It is the fourth most abundant cation in the human body, with an estimated 1 mol of magnesium stored in the body of a normal 70 kg adult, with approximately half of the total physiological magnesium stored in bone tissue. In addition, magnesium is a co-factor for many enzymes, and stabilizes the structures of DNA and RNA. The level of magnesium in the extracellular fluid ranges between 0.7 and 1.05 mmol/L, where homeostasis is maintained by the kidneys and intestine. While serum magnesium levels exceeding 1.05 mmol/L can lead to muscular paralysis, hypotension and respiratory distress, and cardiac arrest occurs for severely high serum levels of 6-7 mmol/L, the incidence of hyper-magnesium is rare due to the efficient excretion of the element in the urine.

The major drawback of magnesium in many engineering applications is its low corrosion resistance, especially in electrolytic, aqueous environments. In body fluids, high chloride concentrations has been reported to lead to high mass losses of magnesium, and magnesium has historically been avoided as a candidate for medical implant material due in large part to the reactivity of the material. Magnesium-based alloys will react with water to produce an ionic form of the material, which is easily removed by the body. While this reactivity/susceptibility to corrosion might be a disadvantage in other applications, it is a desirable characteristic in the present case because this characteristic enables implant components composed of magnesium to be degraded in vivo by the corrosive action of body fluids thereon. The in vivo corrosion of a magnesium-based implant, with the formation of a soluble, non-toxic oxide that is harmlessly excreted in the urine provides an excellent mode by which a bioabsorbable metal component of a medical implant in accordance with the invention can be degraded and absorbed, particularly when considered together with the excellent physical properties of magnesium. Moreover, it has been reported that, due to the functional roles and presence in bone tissue, magnesium may actually have stimulatory effects on the growth of new bone tissue.

In addition to consideration of substantially pure magnesium components, magnesium alloys are also contemplated by the invention as suitable bioabsorbable metal compositions. Control of the compositional make-up of a magnesium alloy allows for the optimization of physical properties and degradation profiles for various uses and requirements for degradable implants. It is important in some applications of the invention to ensure that the magnesium-based implant not corrode too rapidly, as a pure magnesium component would in the physiological pH of 7.4-7.6 and high chloride environment of the physiological system, because excessive corrosion rates could result in the loss of mechanical integrity before it is desired, such as, for example, before surrounding tissue has sufficiently healed. In addition, excessive corrosion rate could result in the production of hydrogen gas as a byproduct of the corrosion process at a rate that is too fast to be dealt with optionally by the host tissue.

Several possibilities exist to tailor the corrosion rate of magnesium. One approach is to use non-toxic, biologically compatible alloying elements to alter its degradation properties. By varying the composition of the alloying elements, one can vary, and thereby control the degradation characteristics of the magnesium alloys. Most alloying elements, such as, for example, aluminum and zinc, are believed to increase the rate of oxidation, while certain alloying elements, such as, for example, rare earth elements, are believed to decrease the oxidation rate of magnesium alloys. In one preferred embodiment, the magnesium-based alloy comprises at least about 85% magnesium by weight, up to about 10% aluminum by weight, up to about 10% zinc by weight and up to about 10% one or more rare earth elements by weight. In a preferred embodiment, the rare earth element or elements are selected from the group consisting of neodymium, cerium, praseodymium, dysprosium and lanthanum. The magnesium alloy can also or alternatively include, for example, manganese, lithium, zirconium, and/or yttrium. One example of a preferred embodiment is an alloy that includes about 2% aluminum, about 1% rare earth metal selected from the group consisting of neodymium, cerium, praseodymium, dysprosium, lanthanum and combinations thereof, and about 97% magnesium. While the proportions of rare earth elements, when present, can vary in an alloy made or selected in accordance with the invention, the rare earth element portion of one exemplary alloy includes about 71% neodymium by weight, about 8% cerium by weight, about 8% dysprosium by weight and about 6% lanthanum by weight. The rare earth portion of another exemplary alloy includes about 51% cerium by weight, about 22% lanthanum by weight, about 16% neodymium by weight and about 8% praseodymium by weight. Another exemplary alloy includes magnesium, aluminum and zinc. Yet another exemplary magnesium-based alloy that can be provided includes magnesium, aluminum and iron. Still another exemplary alloy includes about 95.7% magnesium, about 4% aluminum and about 0.3% manganese, by weight.

In another embodiment, a component of an implant device is composed of a series of layers of different bioabsorbable metal compositions. This orientation can be advantageous, for example, to control the degradation profile of a component. In one embodiment, a component is composed of alternating layers of a highly-reactive bioabsorbable metal composition and a less-reactive bioabsorbable metal compositions. This allows for an iterative rapid degradation of a highly-reactive composition layer followed by slower degradation of the less-reactive layer and so on.

Another exemplary class of metals that are degraded by natural corrosion in vivo by contact with body fluids, and that can be selected for use in accordance with the invention, includes iron and its alloys. Iron-based alloys, in the form of stainless steel, are already widely used in implants. Stainless steel, while primarily iron, is given good corrosion resistance by the addition of chromium in levels above about 15% by weight. Chromium reacts with oxygen at the material surface to form a dense, stable oxide layer that is resistant to corrosion. Operations such as passivation remove reactive iron from the material surface, further improving corrosion resistance.

As an alternative to traditional stainless steel, the present invention contemplates the use of pure iron, substantially pure iron (i.e., iron having a purity of at least about 99%) or an iron-based alloy that will degrade, or corrode, upon contact with body fluids, preferably at a predictable rate. The selected material preferably provides sufficient mechanical strength to provide stabilization for a desired period of time, and then undergoes conversion to an ionic form over time, with subsequent removal from the body. A suitable iron-based alloy can be provided, for example, that includes iron, aluminum and magnesium. In one preferred embodiment the iron-based alloy comprises at least about 85% iron by weight, up to about 10% aluminum by weight and up to about 10% magnesium by weight.

Another mechanism of degradation, which also provides options for predicting and/or controlling the rate of corrosion of a metallic material in vivo, relies on electrochemistry. Another excellent aspect of the invention, therefore, provides a medical implant comprising a component composed of a bioabsorbable metal that is susceptible to electrolytic corrosion. The metal can thereby serve as an anode, which results in corrosion of the metal when current is passed through a circuit that includes the component as an anode. As a result of the corrosion process, the metal degrades and erodes until it is absorbed into the patient's body.

Corrosion can occur actively or passively. In an active corrosion situation, current is actively applied to the metal using an external power source to corrode the metal. In one embodiment of the invention, a power source such as a battery is electrically connected to the bioabsorbable metal composition and is implanted therewith. A wide variety of batteries can be selected for use. One type of battery that can be used to advantage in connection with the invention is one that can be actuated from an external source by non-invasive means. An example of this type of power source can be, for example, a battery such as those used in cardiac pacemakers, neurological devices or other medical implants.

In a passive corrosion process, the oxidation of the metal can be caused by the difference between the electrical potential of the metal and an adjacent metal or solution. For example, galvanic corrosion is caused when two metal parts in electrical contact with one another, or two adjacent metal areas, are at different electrochemical potential. The two metal parts will constitute a galvanic cell, in which the metal part with the lowest electrochemical potential (i.e. the more active metal) will corrode. One exemplary class of metals whose degradation can be influenced by electrochemical means in accordance with the invention includes magnesium and its alloys. Other examples include precious metals such as gold or platinum coupled to stainless steel or cobalt chromium alloys. In addition, where two components are made from the same structural material, one component can be coated with a coating such as a precious metal, thus causing the other component to act as an anode. Also, the two components can be chemically treated in different ways to impart different surface chemistries that would result in different equilibrium potential, thus causing the two components, when they are coupled, to provide a system in which one component acts as an anode and the other acts as a cathode.

A variety of approaches can be employed for achieving different degradation profiles for different device components, or for modifying the degradation profile of a component. For example, and without limitation, it is possible to adjust the relative amounts of ingredients in a metal alloy as discussed above, it is possible to provide a coating over a component to delay the onset of degradation of the underlying bioabsorbable metal, and it is possible to vary the electrochemical environment of a component that degrades by electrochemical corrosion.

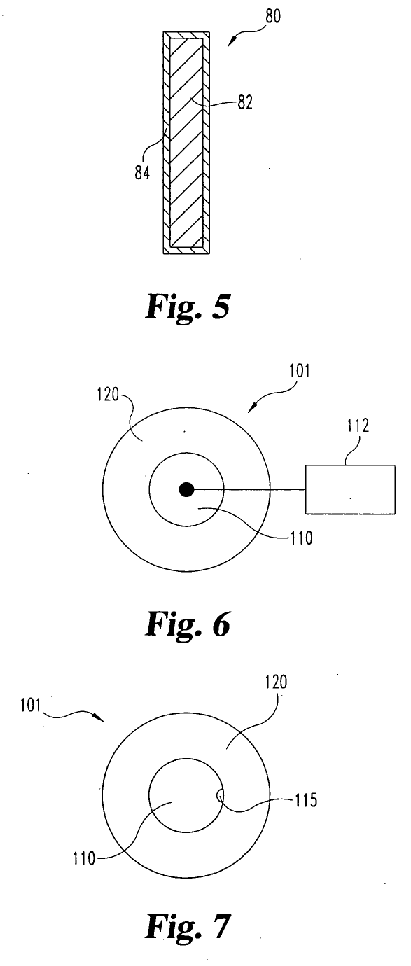

With regard to coatings, the present invention contemplates the use of a structural component that is composed of a bioabsorbable metal, and that is covered by a coating component to control the degradation profile of the structural component. Such a coating can advantageously be composed of a biodegradable composition different than the bioabsorbable metal composition of which the structural component is composed. The coating provides a barrier between the structural component and the body, delaying the corrosion of the bioabsorbable metal composition of which the structural component is composed. A structural component with a coating component thereon is depicted cross-sectionally in FIG. 5, in which spinal rod 80 includes main spinal rod body 82 with coating 84 thereon. The coating can be continuous or discontinuous. In embodiments where the coating is discontinuous (not shown), certain sections of the inner rod are exposed, and such sections will therefore be subject to degradation at an earlier point than coated sections.

Coating 84 can be composed of a second bioabsorbable metal composition in certain preferred embodiments, preferably one exhibiting a relatively low rate of degradation. In other preferred embodiments, coating 84 is composed of a bioabsorbable polymeric composition. A variety of biodegradable polymer compositions can be selected for use in accordance with the present invention, provided that the selected polymer meets the requirements discussed herein. Exemplary biodegradable polymers that can be used include polylactides (also referred to as “poly(lactic acid)”), polycaprolactones (e.g., poly(ε-caprolactone), polyglycolides (also referred to as “poly(glycolic acid)”), polyglyconate, poly-alpha-hydroxy ester acids, polyoxalates, and copolymers thereof, polyurethanes including glucose-based polyurethanes, polycarbonates, including trimethylene carbonate, polyiminocarbonates and tyrosine based polycarbonates, tyrosine based polyarylates and oxalate based polymers and copolymers, such as, for example, isomorphic ploy(hexamethylene co-trans-1,4-cyclohexane dimethylene oxalates). Examples of poly-alpha-hydroxy ester acids include polyhydroxyacetate, polyhydroxybutyrate, polyhydroxyvalerate, and copolymers thereof. Additional biodegradable polymers include poly(arylates), poly(anhydrides), poly ester amides, copoly(ether-ester), polyamide, polylactone, poly(hydroxy acids), polyesters, poly(ortho esters), poly(alkylene oxides), poly(propylene glycol-co fumaric acid), poly(propylene fumerates), polyamides, polyamino acids, polyacetals, poly(dioxanones), poly(vinyl pyrrolidone), biodegradable polycyanoacrylates, biodegradable poly(vinyl alcohols), polyphophazenes, polyphosphonates and polysaccharides, including chitosan. Co-polymers, mixtures, and adducts of any of these polymers may also be employed for use with the invention. Other examples of biodegradable polymers that are well known to those of ordinary skill in the art are described in Biomaterials Science—An Introduction to Materials in Medicine, edited by latner, B. D. et al., Academic Press, (1996). Selection of a particular polymer is based primarily on the known properties of the polymer, such as, for example, the potentiality for cross-linking, polymer strength and moduli, rate of hydrolytic degradation and the like. One of ordinary skill in the art may take these and/or other properties into account in selecting a particular polymer for a particular application.

Persons skilled in the art will also appreciate that polymers selected for use in inventive methods may be manipulated to adjust their degradation rates. The degradation rates of polymers are well characterized in the literature (see Handbook of Biodegradable Polymers, Domb, et al., eds., Harwood Academic Publishers, 1997, the entire contents of which are incorporated herein by reference). In addition, increasing the cross-link density of a polymer tends to decrease its degradation rate. The cross-link density of a polymer may be manipulated during polymerization by adding a cross-linking agent or promoter. After polymerization, cross-linking may be increased by exposure to UV light or other radiation. Co-monomers or mixtures of polymers, for example, lactide and glycolide polymers, may be employed to manipulate both degradation rate and mechanical properties.

In one preferred embodiment, coating 84 is composed of a bioabsorbable composition that degrades relatively slowly and main spinal rod body 82 is composed of a bioabsorbable metal composition that degrades relatively quickly. For example, a polymeric coating could be made to degrade relatively slowly by increasing the degree of cross-linking in the polymer, and a metallic coating can be made to degrade more slowly by altering the selection of alloying elements and/or amounts thereof in the coating. Control of the degradation of the coating could also, of course, be achieved by controlling the thickness of the coating. When the coating is breached at one or more location, body fluids contact the underlying bioabsorbable metal component, and ingress of body fluid to the metal below results in the onset of degradation of the underlying metal. A wide variety of biodegradable metallic or polymeric coatings with different kinetics are contemplated by the invention.

This aspect of the invention can be used to particular advantage in connection with a spinal fusion cage. Specifically, a cage comprising a bioabsorbable metal composition that degrades relatively quickly can be used to provide suitable structural support for a sufficient amount of time by placing a slow-degrading coating component over the structural components that are composed of a fast-degrading bioabsorbable metal. The presence of the coating delays degradation of the underlying bioabsorbable metal until bone-growth into and through the cage has progressed to a point where the structural support of the underlying metal is no longer required. In one embodiment, the coating is effective to protect the bioabsorbable metal composition from degradation for a period of about 6 to 12 months, at which time the underlying metal can be degraded and absorbed relatively quickly, such as, for example, in the following six to twelve months. Of course, the reverse is possible as well. Specifically, a device can include a bioabsorbable metal or polymer composition as a coating layer that degrades relatively quickly, and underlying components that are composed of a slow-degrading bioabsorbable metal.

Another manner of controlling the rate of degradation of a metallic component in accordance with the invention is by varying the electrochemical environment of the component. This can be accomplished, for example, by actuating a source of electrical potential to which the component is electrically connected, by contacting the component to a source of electrical component or by orienting the component in a magnetic field in a manner that results in an electrical potential across the component, to name a few.

In one embodiment, degradation of a component can be prevented by establishing an electrical potential in an implant system that causes the component to operate as a cathode in the system, which will prevent corrosion or degradation of the component. Then, at such time as a surgeon or other medical care provider desires, reversal of the potential would initiate controlled corrosion of the implant by causing the component to become the anode of the system. Control of the electric potential of a component can be achieved, for example, by including a power source component in an implant system, as discussed above.

The present invention also finds advantageous use in connection with implants of which only a portion is to be bioabsorbed. For example, principles of the invention can be advantageously used in applications in which it would be desirable for a structural component of an implant to become more flexible over time. One manner of reducing the stiffness or strength of a construct is by providing a device in which only select components are bioabsorbable, and in which the degradation of such components over time operates to reduce the stiffness of the construct. This principle can be used, for example, to provide a spinal rod whose stiffness decreases over time. Reduction in rod stiffness over time is beneficial because it increases the portion of load being borne by the spine itself over time, thereby resulting in a more robust fusion mass.

In another aspect of the invention, therefore, a spinal rod is provided that includes a main spinal body composed of a non-bioabsorbable metal composition, and one or more region within the cross section of the rod that contain one or more bioabsorbable metal compositions. When implanted, the rod as a whole has a first, relatively high stiffness. With time, the bioabsorbable metal composition or compositions become degraded and are absorbed, yielding a lower stiffness rod. The invention thereby provides a spinal implant useful as spinal stabilization hardware (spinal rod and fixation components) that becomes less stiff with time, requiring the spine and/or fusion mass to carry more load. The additional load on the spine and/or fusion mass is expected to yield a more robust fusion mass.

In one embodiment, a rod with a stiffness that changes over time includes dissimilar metals to create a plural-material rod. One exemplary geometry that could be employed includes a main rod body defining an inner core chamber, and an inner core positioned therein that is composed of a different material. Other potential geometries include a main rod body having multiple interior strands or cores. Of course, the internal cores or strands can be rod-shaped or can take a wide variety of other shapes as would occur to a person of ordinary skill in the art. The internal cores or strands can be made of metals dissimilar to a metal used in the main rod body, thereby causing the cores or strands to act as sacrificial components, dissolving away over time. One or more of the inner cores or strands can optionally be composed of a bioabsorbable polymeric composition.

Various components of implant devices contemplated by the invention can also be composed of non-bioabsorbable polymers or metals. Exemplary non-bioabsorbable, yet biocompatible polymers that can be selected for use include polystyrene, polyesters, polyureas, poly(vinyl alcohol), polyamides, poly(tetrafluoroethylene), and expanded polytetrafluoroethylene (ePTFE), poly(ethylene vinyl acetate), polypropylene, polyacrylate, non-biodegradable polycyanoacrylates, non-biodegradable polyurethanes, mixtures and copolymers of poly(ethyl methacrylate) with tetrahydrofurfuryl methacrylate, polymethacrylate, poly(methyl methacrylate), polyethylene, including ultra high molecular weight polyethylene (UHMWPE), polypyrrole, polyanilines, polythiophene, poly(ethylene oxide), poly(ethylene oxide co-butylene terephthalate), poly ether-ether ketones (PEEK), and polyetherketoneketones (PEKK).

Exemplary non-bioabsorbable, yet biocompatible metals and metal alloys that can be selected for use include titanium and its alloys, zirconium and its alloys, niobium and its alloys, stainless steels, cobalt and its alloys, and mixtures of these materials. In particular embodiments, the metal material includes commercially pure titanium metal (CpTi) or a titanium alloy. Examples of titanium alloys for use include Ti-6Al-4V, Ti-6Al-6V, Ti-6Al-6V-2Sn, Ti-6Al-2Sn-4Zr-2Mo, Ti—V-2Fe-3Al, Ti-5Al-2.5Sn, and TiNi. These alloys are commercially available in a sufficient purity from one or more of the following vendors: ATI Allvac; Timet Industries; Specialty Metals; and Teledyne Wah Chang. In one embodiment, the materials are specifically selected to provide desired load carrying capability with a desired performance characteristics to prevent movement between one or more bony portions or a desired performance characteristic to permit at least some limited movement between adjacent bony portions.

FIG. 6 represents an end view of spinal rod component 101, which includes inner core 110 positioned within main spinal rod body 120. Inner core 110 is composes of a bioabsorbable metal composition, and main spinal rod body 120 is composed of a non-absorbable metal composition. Inner core 110 is preferably coupled to main spinal rod body 120 to enhance the stiffness of the overall rod 101. As used herein, the term “coupled” is intended to mean fused, bonded, interference fit or joined by any other means, whether temporary or permanent. Also shown is optional source 112 of electrical potential operably connected to inner core 110. A person of ordinary skill in the art will understand that source 120 will be included only in embodiments where electrical potential across core 10 is required to achieve degradation of inner core 110 or is desired to increase the rate of degradation of inner core 110.

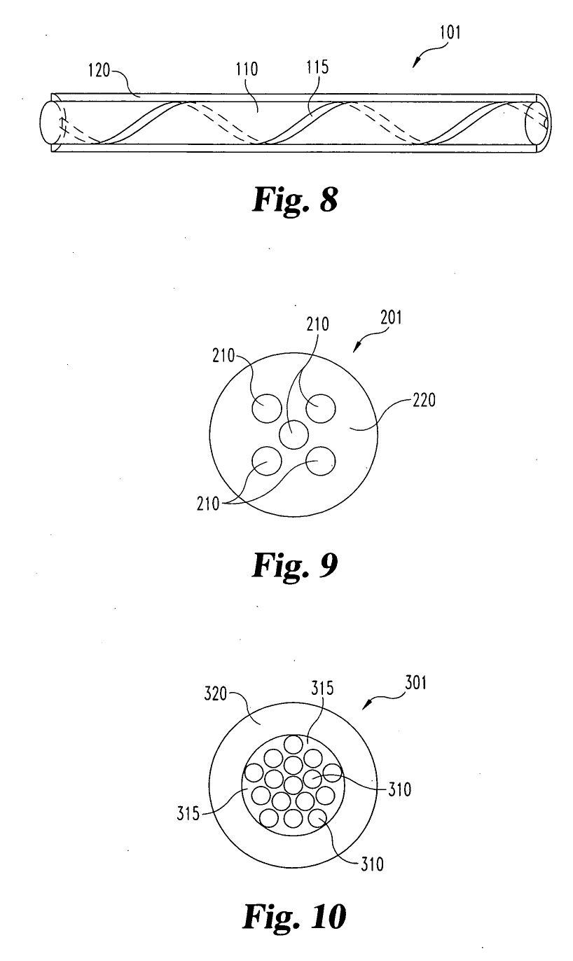

In another embodiment, depicted in FIG. 7, slot 115 is formed between an outer surface of inner core 110 and an inner surface of main spinal rod body 120 to allow infiltration of fluid along the length of rod component 101, to thereby provide for more uniform degradation of inner core 110 after implant. Slot 115 can be formed in an outer surface of inner core 110 or in an inner surface of main spinal rod body 120 (not shown). Slot 115 can extend linearly along a longitudinal axis of rod component 101. Alternatively, slot 115 can be positioned spirally around inner core 110 as depicted in FIG. 8, or in a wide variety of other orientations. The pitch of the spiral can be modified to increase the amount of contact between fluid and inner core 110. While the embodiments depicted in FIGS. 7 and 8 show a single slot, it is intended that the invention also encompass embodiments in which multiple slots, whether longitudinal, spiral or of other design, are provided in a single rod component.

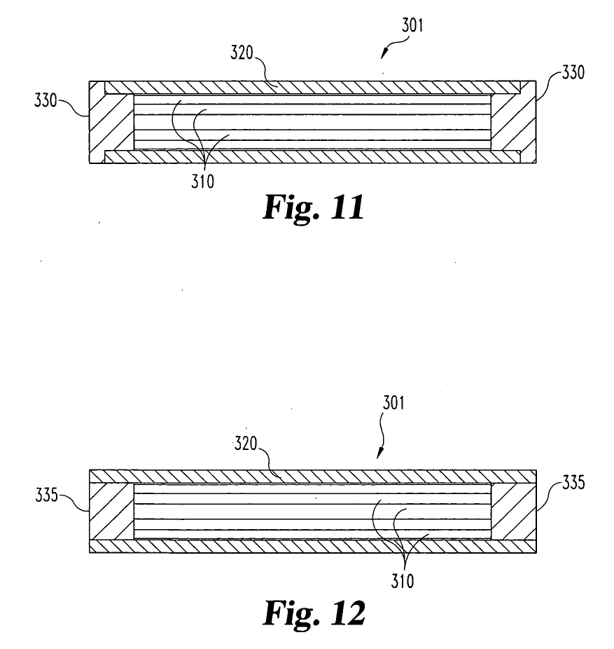

In FIG. 9 an end view of another embodiment is shown. In this embodiment, spinal rod 201 includes a plurality of inner core members 210, also referred to as strands, positioned within main spinal rod body 220. At least one of inner core members 210 is composed of a bioabsorbable metal composition, and main spinal rod body 220 is composed of a non-absorbable metal composition. In other embodiments (not shown), slots are formed between an outer surface of at least one inner core member 210 and a surface of main spinal rod body 220 as the same manner as depicted in FIGS. 7 and 8 in connection with spinal rod component 201, to allow infiltration of fluid along the length of rod component 101, to thereby provide for more uniform degradation of one or more of inner core members 210 after implant. Some or all of members 210 are preferably coupled to main spinal rod body 220. Infiltration of bodily fluids can also or alternatively be provided through one or more holes, which can be formed in various sizes, drilled from the outside surface of the rod and extending radially to inner core member 210 (not shown). The location and diameter of such holes could in turn control the degradation location of the inner members as well as the kinetics of degradation. In addition, one or more caps, seals or plugs (not shown) can be used to shield one or more of core members 210 from bodily fluids for an initial period of time, in the same manner as described below in connection with the caps 330 and plugs 335 depicted in FIGS. 11 and 12.

In another embodiment, depicted cross-sectionally in FIG. 10, spinal rod component 301 includes a tubular main spinal rod body 320 defining a space within which a plurality of inner rods 310, also referred to as “core members” or “strands,” are positioned. At least one of inner rods 310 is composed of a bioabsorbable metal composition, and main spinal rod body 320 is composed of a non-absorbable metal composition. In the embodiment depicted in FIG. 10, inner rods 310 are shaped in a manner whereby voids 315 are created between individual inner rods 310 and/or between inner rods 310 and main spinal body 320. One potential benefit of voids 315 is that a high degree of fluid contact with bioabsorbable elements would be expected due to increased available space for fluid penetration within rod component 301, which would be expected to increase the rate of degradation of biodegradable inner rods. In other embodiments, voids 315 can be filled with a bioabsorbable composition, such as a bioabsorbable metal or a bioabsorbable polymer, to provide another mechanism of control of the rate of degradation.

In the embodiment depicted in partial longitudinal cross section view in FIG. 11, inner rods 310 are shorter than main rod body 320, and caps or seals composed of a bioabsorbable composition are positioned at and engaged to the ends of main rod body 320 to operate as bioabsorbable caps, or seals, 330. A person skilled in the art will recognize that, upon breach of caps after implant of the device via natural degradation and bioabsorption processes, body fluids enters voids 315 and degradation and bioabsorption processes begin to act on inner rods 310 that are composed of bioabsorbable metal compositions. Embodiments employing bioabsorbable caps can be modified to control the time frame within which fluid will contact inner rods 310. For example, the occurrence of cap breach can be delayed by increasing the thickness of caps 330, or by selecting caps 330 that are composed of bioabsorbable composition having a longer degradation profile. It is not intended that the invention be limited by any particular shape of cap 330. For example, in another embodiment depicted in FIG. 12, main spinal rod body 320 is capped using a plug 335 that can be positioned entirely within main spinal rod body 320. Caps 330 or plugs 335 and main rod body 320 can optionally be cooperatively threaded for positive engagement to one another if desired (not shown)

The invention also contemplates embodiments in which main spinal rod body 120, 220, 320 is also composed of a bioabsorbable metal composition, albeit one having a degradation profile featuring a relatively later onset of degradation or with a significantly slower degradation rate. This can be achieved, for example by using distinct electrochemical means within the device to selectively degrade different components at different times. For example, it might be desirable to position electrically insulative layers between various components to achieve the desired selective degradation of adjacent metallic components. Alternatively, the electrical degradation susceptibility of the respective bioabsorbable metal compositions can be controlled to ensure that a given electric potential across connected components is effective to degrade one component before another. Alternatively, coatings can be used to control the degradation profiles of the respective components, as discussed above. It is understood that a coating used to delay the degradation of a main spinal rod body 120, 220, 320 will preferably extend between the main spinal rod body 120, 220, 320 and inner core 110 and inner core members 210, 310, respectively (not shown). Such a device can be designed, for example, to proceed through a first phase during which flexibility increases, and then proceed through a second phase during which the entire device is degraded and absorbed.

In another manner of increasing the flexibility of a spinal rod over time, an electrochemical degradation process effective to degrade a unitary rod can be allowed to continue for a period of time effective to remove only a portion of the metal of the rod, at which time it is halted by removing the electric potential or returning it to its original state. The degradation process can be stopped, for example, to retain a construct of reduced stiffness, thereby increasing loading of an associated fusion mass. Of course, the degradation process can be resumed at a later time if desired, potentially in several steps, to achieve incremental increases in the loading of the fusion mass, or to degrade and absorb the material entirely.

Another excellent use of the principles of the present invention is in devices for providing dynamizable translations to orthopedic implants as described in U.S. Patent Application Publication No. 2005/0085812, which is incorporated herein by reference in its entirety. Briefly, this patent application describes another type of device that can provide initial, more rigid, support and/or fixation of selected bone structures and, after a selected period of time or under certain conditions, the amount and nature of the support/fixation can vary to facilitate a desirable treatment. The biodegradable component in such a device operates as a restricting component for the device, which can provide rigidity and support for both the implanted orthopedic fusion device and, consequently, the attached bone structures.

Such a device for providing dynamizable translations can operate by having a main support structure composes of a non-bioabsorbable metal composition (or a bioabsorbable metal composition with a relatively later onset or lower rate degradation profile) that includes slots or other apertures (referred to herein as “apertures) through which screws, fasteners or other anchors can be fastened to adjacent boney elements such as, for example adjacent vertebrae, to provide dynamic (i.e., movable) fixation of the support structure to the boney elements. The movement can be restricted initially by the presence of a restricting component in the aperture that is effective to initially prevent movement of the anchors within the aperture. The restricting component is composed of a bioabsorbable metal composition. This allows the fixation device to become dynamizable, or change its support characteristics in vivo upon degradation of the restricting component or components. This change in support characteristics can be important for developing strong, new bone tissue at the bone defection or fusion site. This prevents stress shielding of the new bone ingrowth and minimizes the risk for the development of pseudoarthrodesis.

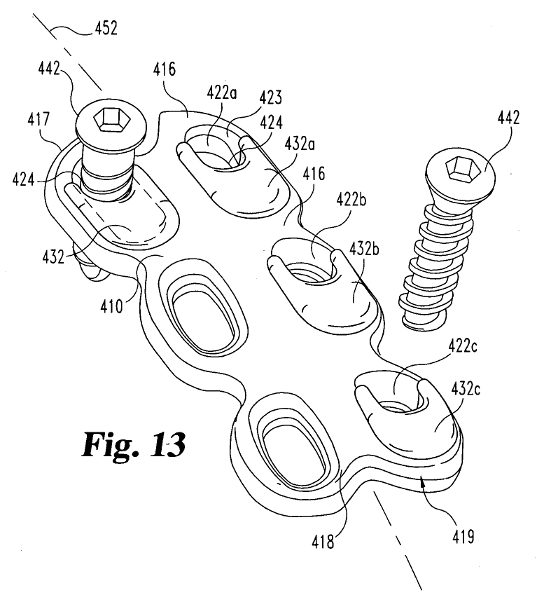

FIG. 13 is a perspective view of one embodiment of an orthopedic device 410 comprising an elongate member 416 defining an elongate axis 452. In the illustrated embodiment, member 416 comprises a bone plate 418. Device 410 can include one receptacle 424 or a plurality of receptacles 422a, 422b, 422c. Bone fastener 442 can be inserted through receptacle 424 to secure elongate member 416 to one, two, or more bone portions. In a preferred embodiment, one or more of receptacles 424, 422a, 422b, 422c and the like are sized to have a larger opening than the outer diameter of the threads and/or shank of fastener 442.

Restricting component 432 is operatively positioned within receptacle 424 such that it further restricts the translational and/or rotational motion of attached bone portions. Receptacles 424, 422a, 422b, 422c and the like can be configured to allow or restrict movement of secured bone portions in only one direction, or two or more directions, as desired. Similarly, receptacles 424, 422a, 422b, 422c and the like can be configured to allow either rotation or translation or both, as desired. Additional restricting components 432a, 432b, 432c can optionally be included if desired.

Restricting components 432, 432a, 432b, 432c are composed of a bioabsorbable metal composition as described herein. In a preferred embodiment, after restricting component 432 has been eliminated, fastener 442 continues to secure elongate member 416 to attached bone portions. Elongate member 416 continues to provide at least some support to attached bone and to restrict at least some of the translational and/or rotational motion of attached bone portions.

Components described herein as being composed of a bioabsorbable metal composition can alternatively be composed of a composite including a bioabsorbable metal and a second bioabsorbable material, such as for example a bioabsorbable polymeric material or a bioactive material such as hydroxyapatite, ACP, BMP or other osteoconductive or osteoinductive material. When the invention is practiced using a composite of a bioabsorbable metal and a bioactive material, once implanted the composites would initially have strength and ductility comparable to the bone being treated, would retain these properties for a sufficient period of time for the bone to heal, and then would undergo degradation, absorption, and/or excretion. In addition, in a preferred embodiment, the bioabsorbable metal is biodegradable at a rate consistent with regeneration or remodeling of the surrounding tissue. Implants formed of or made from bioabsorbable metals and including a bioactive agent that induces healing, such as, for example, bone or a bone derivative, advantageously provide good structural support while also promoting a mechanism of healing that includes remodeling of the bioactive agent and then transformation of the bioabsorbable metal.

In another embodiment, at least one component of the device comprises a bioactive material such as an osteoconductive or osteoinductive bioactive material. In one exemplary manner of including the bioactive material, it is impregnated in the component. A bioabsorbable metal can be compounded with the bioactive material to produce a component having the bioactive material impregnated therein or, alternatively, a component can be manufactured to be porous, and a bioactive material can then be impregnated therein. Alternatively, the component can be surface treated with the bioactive material.

The term “bioactive material” (also referred to herein as “bioactive agent”), is used herein to refer to a substance or other composition of matter that has an effect on living tissues or that alters, inhibits, activates, or otherwise affects biological or chemical events such as, for example, a composition that promotes an immune response, promotes cell proliferation, or has some other effect. In certain preferred embodiments, the bioactive material is effective to promote host tissue integration, such as, for example, ingrowth of bone, after surgical implantation of the composite material in a patient.

A wide variety of bioactive materials can be selected for use in accordance with the present invention. For example, bioactive agents may include, but are not limited to osteogenic, osteoinductive, and osteoconductive agents, anti-AIDS substances, anti-cancer substances, antibiotics, immunosuppressants (e.g., cyclosporine), anti-viral agents, enzyme inhibitors, neurotoxins, opioids, hypnotics, anti-histamines, lubricants, tranquilizers, anti-convulsants, muscle relaxants and anti-Parkinson agents, anti-spasmodics and muscle contractants including channel blockers, miotics and anti-cholinergics, anti-glaucoma compounds, anti-parasite, anti-protozoal, and/or anti-fungal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, targeting agents, neurotransmitters, proteins, cell response modifiers, and vaccines. In a certain preferred embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals,” Edited by Susan Budavari et al., CRC Press, 1996, the United States Pharmacopeia-25/National Formular-20, published by the United States Pharmcopeial Convention, Inc., Rockville Md., 2001, and the “Pharmazeutische Wirkstoffe,” edited by Von Keemann et al., Stuttgart/N.Y., 1987, all of which are incorporated herein by reference. Drugs for human use and drugs for veterinary use listed by the FDA in the Code of Federal Regulations, all of which is incorporated herein by reference, are also considered acceptable candidates for use in accordance with the present invention.

In certain preferred embodiments, the bioactive agent is a biomolecule or comprises a biomolecule. The term “biomolecule,” as used herein, refers to a class of molecules (e.g., proteins, amino acids, peptides, polynucleotides, nucleotides, carbohydrates, sugars, lipids, nucleoproteins, glycoproteins, lipoproteins, steroids, lipids, etc.) that are commonly found in cells and tissues, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active subunits. In some embodiments, the biomolecule is a growth factor, cytokine, extracellular matrix molecule or a fragment or derivative thereof, for example, a cell attachment sequence such as RGD. Bioactive agents selected for use in accordance with the invention include synthetic bioactive agents and bioactive agents that are isolated or derived from natural sources. Examples of preferred bioactive agents include bone, bone morphogenic protein and growth factors including for example transforming growth factor-β.

In one preferred embodiment, the bioactive material is a particulate material having an average particle size of up to about 80 microns. One preferred bioactive agent comprises bone particles milled from whole bone or bone sections. As used herein, the term “bone” is intended to refer to bone recovered from any source including animal and human, for example, human bone recovered for the production of allografts, and animal bone recovered for the production of xenografts, such allografts and xenografts suitable for implantation into a human. Such bone includes: any bone or portion thereof, including cut pieces of bone, including cortical and/or cancellous bone, for example, recovered from a human including a living human or a cadaver, or animal, and processed for implantation into a living patient. Such bones include for example: the humorous, hemi-pelvi, tibia, fibula, radius, ulna, rib, vertebrae, mandibular, femur, and ilia, and any cut portion thereof. Such bone may be demineralized or not demineralized. The bone can be demineralized or non-demineralized in alternate embodiments. Reduction of the antigenicity of allogeneic and xenogeneic tissue can be achieved by treating the tissues with various chemical agents, e.g., extraction agents such as monoglycerides, diglycerides, triglycerides, dimethyl formamide, etc., as described, e.g., in U.S. Pat. No. 5,507,810, the contents of which are incorporated by reference herein.

The bioactive agent can comprise either intact extracellular matrix or its components, alone or in combination, or modified or synthetic versions thereof. Exemplary extracellular matrix components include but are not limited to collagen, laminin, elastin, proteoglycans, reticulin, fibronectin, vitronectin, glycosaminoglycans, and other basement membrane components. Various types of collagen (e.g., collagen Type I, collagen Type II, collagen Type IV) are suitable for use with the invention. Collagens may be used in fiber, gel, or other forms. Sources for extracellular matrix components include, but are not limited to, skin, tendon, intestine and dura mater obtained from animals, transgenic animals and humans. Extracellular matrix components are also commercially available.

The following definitions and meanings are also considered pertinent in reading the descriptions in the present specification.

“Polynucleotide,” “nucleic acid” or “oligonucleotide”: The terms “polynucleotide,” “nucleic acid” or “oligonucleotide” refer to a polymer of nucleotides. The terms “polynucleotide,” “nucleic acid” and “oligonucleotide” may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thihymidine, inosine, pyrrolo-pyrimidine, 3-methyl adenosine, C5-propynylcytidine, C5-propynyluridine, C5-bromouridine, C5-fluorouridine, C5-idouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as siRNA.

“Polypeptide,” “peptide” or “protein”: As used herein, a “polypeptide,” “peptide” or “protein” includes a string of at least two amino acids linked together by peptide bonds. The terms “polypeptide,” “peptide” and “protein” may be used interchangeably. Peptide may refer to an individual peptide or a collection of peptides. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide,” as used herein, refer to any polymer or oligomer of carbohydrate residues. The polymer or oligomer may consist of anywhere from two to hundreds to thousands of sugar units or more. “Oligosaccharide” generally refers to a relatively low molecular weight polymer, while “starch” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., phosphorylated, cross-linked). Carbohydrate polymers or oligomers may include natural sugars (e.g., glucose, fructose, galactose, mannose, arabinose, ribose, and xylose) and/or modified sugars (e.g., 2′-fluororibose, 2′-deoxyribose, and hexose). Polysaccharides may also be either straight or branch-chained. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

Other materials can also be included in a composite structure made or selected in accordance with the invention, such as, for example, ingredients to increase the stability or shelf life of any bioactive agent included in the composite, or a buffer, which can provide an advantageous effect after a composite material containing certain biodegradable polymers is implanted. As certain biodegradable polymers undergo hydrolysis in the body, acidic degradation products formed may be implicated in irritation, inflammation, and swelling (sterile abscess formation) in the treated area. To counteract this effect, a neutralization compound, or buffer, can be included in the biodegradable material to neutralize the acidic degradation products and thereby reduce the sterile abscess reaction.

In addition to ameliorating the rate of decline in pH in the region of polymer hydrolysis, the use of hydroxyapatite also supports osteoconductivity. Thus, HA not only promotes bony ingrowth, but also acts as a buffer thereby preventing the formation of sterile abscesses that have been attributed to the acidic degradative products of PLGA implants.

As will be appreciated by a person of ordinary skill in the art, the final performance of a device or component made in accordance with the invention is influenced by its degradation rate and mechanism, component porosity, activity of any bioactive agent present and component mechanical properties including strength, fracture toughness, and modulus. While bioabsorbable metals and polymers formed as a solid mass will typically degrade from the surface in, penetration of cells and/or body fluids into the interior of the device or component can increase the overall degradation rate and cause more uniform degradation across a cross-section thereof, where desired. Both the inherent porosity of the device or component (if any) and induced pathways influence the overall degradation rate by facilitating the infiltration of cells and fluid into the composite.

The present invention contemplates modifications as would occur to those skilled in the art without departing from the spirit of the present invention. In addition, the various procedures, techniques, and operations may be altered, rearranged, substituted, deleted, duplicated, or combined as would occur to those skilled in the art. All publications, patents, and patent applications cited in this specification are herein incorporated by reference as if each individual publication, patent, or patent application was specifically and individually indicated to be incorporated by reference and set forth in its entirety herein.

Any reference to a specific direction, for example, references to up, upper, down, lower, and the like, is to be understood for illustrative purposes only or to better identify or distinguish various components from one another. Any reference to a first or second vertebra or vertebral body is intended to distinguish between two vertebrae and is not intended to specifically identify the referenced vertebrae as adjacent vertebrae, the first and second cervical vertebrae or the first and second lumbar, thoracic, or sacral vertebrae. These references are not to be construed as limiting any manner to the medical devices and/or methods as described herein. Unless specifically identified to the contrary, all terms used herein are used to include their normal and customary terminology. Further, while various embodiments of medical devices having specific components and structures are described and illustrated herein, it is to be understood that any selected embodiment can include one or more of the specific components and/or structures described for another embodiment where possible.

While the invention has been described in detail in the foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a,” “an,” “at least one” and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. An orthopedic implant device, comprising a bioabsorbable metal composition, and comprising at least two different components that exhibit different degradation profiles after implant.

2. The device in accordance with claim 1 wherein the device comprises at least two structural components that are composed of different bioabsorbable metal compositions that exhibit different absorption profiles after implant.

3. The device in accordance with claim 1 wherein the device comprises at least two structural components that are composed of a bioabsorbable metal composition, at least one of which has a coating thereon composed of a bioabsorbable composition; wherein the structural components exhibit different absorption profiles after implant due to the presence of the coating.

4. The device in accordance with claim 3 wherein at least two of the structural components are composed of the same bioabsorbable metal composition.

5. The device in accordance with claim 3 wherein at least two of the structural components have different coatings thereon, each coating composed of one or more bioabsorbable composition; and wherein the components exhibit different absorption profiles under a given set of conditions due to the presence of the coatings.

6. The device in accordance with claim 3 wherein at least two of the structural components comprise different bioabsorbable metal compositions.

7. The device in accordance with claim 1 wherein said device comprises at least one bone-engaging element and at least one non-bone-engaging element, and wherein the absorption profiles of the bone-engaging element and the non-bone-engaging element are selected to ensure that absorption of the non-bone-engaging element is completed before the occurrence of significant degradation of the bone-engaging element.

8. The device in accordance with claim 7 wherein said bone-engaging element is selected from the group consisting of a bone screw and an anchor; and wherein said non-bone-engaging element is selected from the group consisting of a rod, a bracket and a plate.

9. The device in accordance with claim 1 wherein the device comprises at least two different metals, and wherein at least one of the metals is a bioabsorbable metal composition.

10. The device in accordance with claim 9, wherein the device includes a spinal rod that comprises:
a main spinal rod body, the main spinal rod body composed of a first metal and defining at least one internal chamber; and
a core positioned within the internal chamber, the core composed of a second metal;
wherein the second metal is a bioabsorbable metal.

11. The device in accordance with claim 10 wherein the first metal is a non-absorbable metal.

12. The device in accordance with claim 11 wherein the first and second metals are selected such that at least a portion of the core dissolves away over time after the device is implanted.

13. The device in accordance with claim 10, further comprising a source of electrical potential operably connected to said core.

14. The device in accordance with claim 13 wherein said source is a battery.

15. The device in accordance with claim 10 wherein an electrochemical potential exists between the first metal and the second metal.

16. The device in accordance with claim 10, further comprising a metallic coating positioned between the main spinal rod body and the core, the metallic coating comprising a third metal, wherein an electrochemical potential exists between the second metal and the third metal.

17. The device in accordance with claim 10 wherein the core is coupled to the main spinal rod body.

18. The device in accordance with claim 10 wherein a channel is formed between a surface of the main spinal rod body and the core to provide a conduit for infiltration of body fluid after implantation.

19. The device in accordance with claim 10 wherein the main spinal rod body defines at least one radial aperture between first and second ends of the main spinal rod body, the aperture effective to provide a conduit for infiltration of body fluid into contact with the core after implantation

20. The device in accordance with claim 10 wherein the main spinal rod body defines a plurality of internal chambers, and wherein the device comprises a plurality of core members positioned within some or all of the internal chambers.

21. The device in accordance with claim 20 wherein said plurality of cores comprises at least two core members composed of different bioabsorbable metal compositions.

22. The device in accordance with claim 21 wherein the different bioabsorbable metal compositions have different degradation profiles.

23. The device in accordance with claim 20 wherein said device further comprises a plurality of caps, plugs or seals operable to shield at least one of said core members from contact with body fluid after implant.

24. The device in accordance with claim 20 wherein the caps, plugs or seals are composed of a bioabsorbable material.

25. The device in accordance with claim 20 wherein a first channel is formed between a first surface of the main spinal rod body and a first core member to provide a first conduit for infiltration of body fluid, and a second channel is formed between a second surface of the main spinal rod body and a second core member to provide a second conduit for infiltration of body fluid.

26. The device in accordance with claim 10 wherein the main spinal rod body defines an internal chamber, and wherein the device comprises a plurality of core members positioned within the internal chamber.

27. The device in accordance with claim 26 wherein said plurality of core members comprises at least two core members composed of different bioabsorbable metal compositions.

28. The device in accordance with claim 27 wherein the different bioabsorbable metal compositions have different degradation profiles.

29. The device in accordance with claim 26 wherein at least one channel is formed adjacent a first surface of a first core member and a second surface of a second core member to provide a conduit for infiltration of body fluid after implantation.

30. The device in accordance with claim 1 wherein the device comprises:
an elongate member including a receptacle therein configured to be fixedly secured to two or more bone portions allowing translational or rotational, or both translational and rotational movement of a first one of the bone portions relative to a second one of the bone portions; and
a restricting component composed of a bioabsorbable metal composition and disposed in the receptacle to inhibit the translational, the rotational, or both the translational and rotational movement of the first of the bone portions relative to the second of the bone portions.

31. The device in accordance with claim 30 wherein said elongate member allows limited translational, or rotational, or translational and rotational movement of the first of one of said two or more bone portions relative to the second of said two or more bone portions after the restricting component biodegrades.

32. The device in accordance with claim 1 wherein degradation of the bioabsorbable metal composition is initiated or enhanced by an electrical potential across the bioabsorbable metal composition.

33. The device in accordance with claim 32 wherein the device comprises at least two components composed of different metals in contact with one another; and wherein the electrical potential results from an electrochemical potential between the two metals.

34. The device in accordance with claim 32, further comprising an electrical potential source operably connected to the bioabsorbable metal composition.

35. The device in accordance with claim 1 wherein all components of the device are absorbed within a five-year period.

36. The device in accordance with claim 1 wherein at least one component of the device comprises a bioactive material.

37. The device in accordance with claim 36 wherein the bioactive material is impregnated in the component.

38. The device in accordance with claim 36 wherein the component is surface treated with the bioactive material.

39. The device in accordance with claim 36 wherein at least one component comprises a bioabsorbable metal compounded with the bioactive material.

40. The device in accordance with claim 36 wherein at least one component comprises a porous bioabsorbable metal having a biological material impregnated therein or coated thereon.

41. The device in accordance with claim 1 wherein at least one component comprises a bioabsorbable metal compounded with a bioabsorbable polymer.

42. An orthopedic implant device comprising at least one structural component constructed from a bioabsorbable metal composition, the structural component having physical properties effective to withstand tensile loads, torsional loads and bending loads encountered during spinal implant procedures and during a first period of time of at least 6 months post-implant, and the structural component being absorbed within a second period of time.

43. The device in accordance with claim 42 wherein the first and second periods of time begin when the device is surgically implanted; wherein the first period of time ends after bone repair or fusion has proceeded to a degree where the physical properties of the component are no longer required; and wherein the second period of time is greater than the first period of time.

44. The device in accordance with claim 43 wherein the second period of time is less than three years.

45. The device in accordance with claim 42 wherein the bioabsorbable metal composition comprises a member selected from the group consisting of magnesium, a magnesium-based alloy, iron and an iron-based alloy.

46. The device in accordance with claim 45 wherein the bioabsorbable metal composition is a magnesium-based alloy comprising at least about 85% magnesium by weight and an alloying element portion comprising an element selected from the group consisting of aluminum, zinc, a rare earth element, manganese, lithium, zirconium and yttrium.

47. The device in accordance with claim 45 wherein the bioabsorbable metal composition is an iron-based alloy comprising at least about 85% iron by weight and an alloying element portion comprising an element selected from the group consisting of aluminum and magnesium.

48. The device in accordance with claim 42 wherein said device further comprises a coating component effective to prevent degradation of the bioabsorbable metal composition during some or all of the first period of time; wherein breach of the coating initiates degradation of the structural component.

49. The device in accordance with claim 48 wherein the coating comprises a bioabsorbable composition.

50. The device in accordance with claim 49 wherein the bioabsorbable composition is selected from the group consisting of a bioabsorbable metal composition and a bioabsorbable polymeric composition.

51. The device in accordance with claim 49 wherein the bioabsorbable composition comprises a bioactive agent impregnated therein.

52. The device in accordance with claim 49 wherein the bioabsorbable composition is selectively degradable, thereby providing for controlled removal of the coating and initiation of degradation of the component.

53. The device in accordance with claim 52 wherein degradation of the coating is initiated by application of an electrical current to the coating.

54. An orthopedic implant device, comprising:
at least one means constructed from a bioabsorbable metal composition for reliably bearing tensile loads, torsional loads and bending loads encountered during normal post-spinal implant activity for a first period of time, and for becoming degraded and absorbed during a second period of time that ends when the bearing means is fully absorbed; and
at least one means constructed from a bioabsorbable metal composition for engaging said bearing means with a bone for a third period of time, for reliably bearing tensile loads, torsional loads and bending loads encountered during the spinal implant procedure, and for becoming degraded and absorbed during a fourth period of time that ends when the engaging means is fully absorbed.

55. The device in accordance with claim 54 wherein said first, second, third and fourth periods of time all begin when said device is surgically implanted; and wherein said third period of time is at least as long as the second period of time.

56. An orthopedic implant product, comprising:
at least two different components that exhibit different degradation profiles under a given set of conditions, one of said components composed of a bioabsorbable metal composition, wherein degradation of the bioabsorbable metal composition is initiated or enhanced by an electrical potential applied to the bioabsorbable metal composition; and
instructions, recorded in a tangible medium, regarding applying an electrical potential across the bioabsorbable metal composition.

57. The product in accordance with claim 56 wherein the instructions include instructions for determining an appropriate time to apply the electrical potential to synchronize the initiation of degradation to a desired stage of healing or fusion.

58. A method for treating a bone defect comprising fixedly attaching the device of claim 1 to two or more bone portions.

59. A method for treating a bone defect, comprising:
providing an orthopedic implant device comprising a bioabsorbable metal composition, and comprising at least two different components that exhibit different degradation profiles under a given set of conditions;
securing the device to first and second bone portions; and
allowing the biodegradable metal composition to degrade in vivo.
